(19) United States
(12) Patent Application Publication
(10) Pub. No.: US 2014/0336141 A1
(43) Pub. Date: Nov. 13, 2014

(54) MAO-B INHIBITORS DERIVED FROM THE ONE-CARBON CYCLE

(71) Applicant: RANDOX LABORATORIES LTD., Crumlin (GB)

(72) Inventors: Maria Zellner, Vienna (AT); Ellen Umlauf, Vienna (AT)

(21) Appl. No.: 14/367,498

(22) PCT Filed: Dec. 21, 2012
(86) PCT No.: PCT/EP2012/076815

§ 371 Date: Jun. 20, 2014

(30) Foreign Application Priority Data
Dec. 23, 2011 (GB) 1122227.0

(51) Int. Cl. A61K 31/7076; C12Q 1/68; A61K 31/519; A61K 31/4415; A61K 31/714

(52) U.S. Cl. CPC A61K 31/7076; A61K 31/4415; A61K 31/714; A61K 31/519; C12Q 1/6881; C12Q 2600/136
USPC 514/46; 514/345; 514/52; 514/249; 435/6.11

(57) ABSTRACT

The invention exploits the elucidation of a connection between the S-adenosylmethionine one-carbon cycle and MAO-B levels and describes drug efficacy testing methods and the use of compounds of the S-adenosylmethionine one-carbon cycle as MAO-B inhibitors in disease prevention and treatment.

### BACKGROUND

### Alzheimer's Disease

Late-onset Alzheimer's disease (LOAD) is the most prominent form of dementia among the elderly (>65 yrs). The only known genetic risk factor is the Apolipprotein E4 allele. In contrast to the early-onset variant, LOAD patients do not carry any mutations in the genes of amyloid precursor protein (APP), presenilin 1 and presenilin 2. The disease is characterized by progressive cognitive dysfunction, and various behavioural and neuro-psychiatric disturbances such as agitation, psychosis, depression, apathy, disinhibition, anxiety, purposeless behaviour, and disorders of sleep and appetite. Histopathological hallmarks in the brain are extracellular amyloid plaques and neurofibrillary tangles in neurons. Amyloid plaques are formed by aggregation of amyloid beta (1-42) that is derived from proteolytic processing of APP by beta and gamma secretases. Neurofibrillary tangles are found in cell bodies and apical dendrites as neurofibrillary tangles, in distal dendrites as neuropil threads, and in the abnormal neurites that are associated with some plaques (neuritic plaques).They are mainly composed of hyperphosphorylated aggregates of the protein tau.

### DNA Methylation and Alzheimer's Disease

Approximately 70% of annotated gene promoters are associated with CpG islands, including most of the housekeeping genes, many tissue-specific genes and developmental regulator genes. CpG islands are sites of transcriptional initiation and CpG island methylation leads to silencing of the associated promoter, and to decreased expression levels of the respective protein. Global DNA hypomethylation has been observed in the entorhinal cortex of LOAD patients. It has also been shown that several proteins such as presenilin 1 and APP that are involved in the beta amyloid formation in LOAD are regulated by promoter methylation. Additionally, APOE, the only known genetic LOAD-risk factor, and genes involved in methylation homeostasis (MTHFR, DNMT1) show a significant inter-individual epigenetic variability in LOAD, which may contribute to LOAD predisposition.

### The One-Carbon Cycle Methylation by SAM

DNA methyltransferases (DNMTs) use S-adenosylmethionine (SAM), the main methyl donor in eukaryotes, as substrate for methylating cytosines on genomic DNA. The resulting S-adenosylhomocysteine (SAH) is a competitive inhibitor of DNMTs. SAH is hydrolyzed to adenosine and homocysteine, which can be re-methylated to methionine. These compounds are connected via the One-carbon cycle which is the only means of SAM production (FIG. 1).

### The One-Carbon Cycle and Alzheimer's Disease

Administration of folic acid, vitamin B12 and vitamin B6 to AD patients with mild cognitive impairment for two years reduced brain atrophy and improved cognitive function when compared to the placebo group (1). Vitamin B12 therapy significantly reduced platelet MAO activity (Mao-B is the predominant isoform in platelets) in LOAD patients to apparently normal levels (2). The Inventors showed that increased meat consumption improves cognitive functions in young volunteers and reduces the platelet Mao-B level and that platelet Mao-B concentration inversely correlates with the plasma level of vitamin B12, which is a cofactor in the One-carbon cycle (3).

### MAO-B, MAO-B Inhibitors, Alzheimer's Disease and Other Pathologies

The flavoenzyme monoamine oxidase B (MAO-B) is located in the outer membrane of mitochondria and catalyses the oxidative deamination of amine neurotransmitters and dietary amines using O₂ as the electron acceptor. MAO-B preferentially degrades phenylethylamine and benzylamine, as well as the cognition enhancing neurotransmitter dopamine. Oxidative deamination of primary monoamines by MAO-B results in the species with proven or potential toxicity, namely ammonia, aldehydes and hydrogen peroxide. Furthermore, MAO-B inhibition reduces dopamine inactivation enabling the neurotransmitter's increased availability in dopaminergic neurons. The activity of MAO-B in brain and in platelets is significantly increased in LOAD (EP1891445) and it has been demonstrated that MAO-B mRNA and MAO-B protein levels are elevated in platelets of LOAD patients (PCT/GB2010/052023). MAO-B inhibitor drugs currently find use in the treatment of hypertension, Parkinson's disease, senile dementia and depression. Thus evidence suggests the potential use of MAO-B inhibitors for the treatment of AD.

The search for therapeutic drugs for the treatment of debilitating pathologies is an ongoing and relentless process. However, it is also a lengthy and extremely costly exercise, with most candidate drug development programs being terminated before drug approval. The elucidation of possible pathophysiological pathways is an extremely difficult but highly desirable goal as it enables a more targeted approach to candidate drug choice, expedites the drug development process and increases the chances of a drug being approved for therapy. Diseases such as hypertension, depression, Parkinson's disease and dementias can be treated using MAO-B inhibitors. Described herein are novel compounds of the MAO-B inhibitor class identified through the elucidation of the connection of MAO-B to the One-carbon cycle.

### REFERENCES

(1) Smith A. D. et al. (2010). PLoS One, 8; 5(9): e12244.

(2) Regland B. et al. (1991). Eur. Arch. Psychiatry Clin. Neurosci., 240(4-5): 288-91.

(3) Zellner M. et al. (2011). J Neural Transm., 118(5): 653-62.

### FIGURES

FIG. 1 One-carbon cycle of SAM production

FIG. 2 Relative % MAO-B levels in cells treated with SAM, vitamin 6, vitamin 12 and control

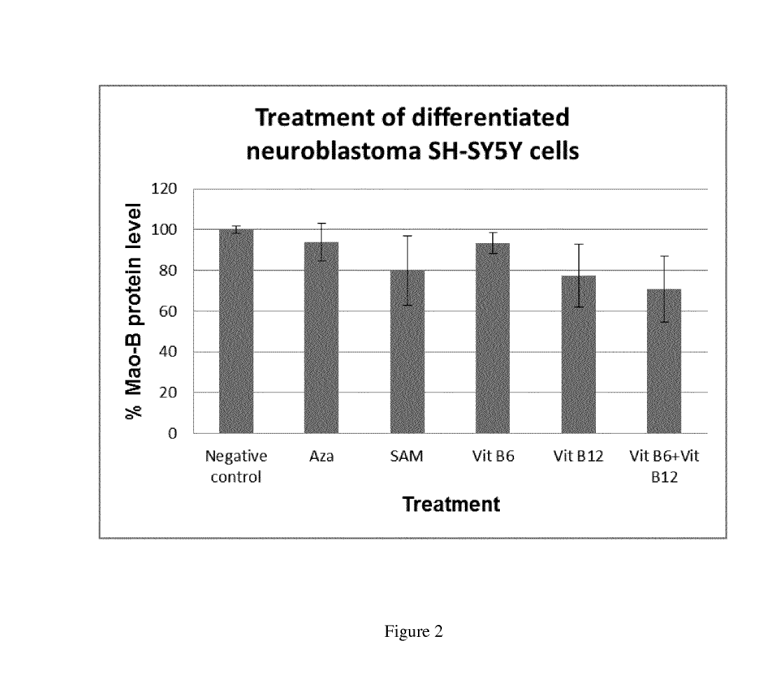

FIG. 3 Results of experiment treating Hep2G cells with 5-aza-2-deoxycytidine in relation to MAO-B expression levels normalised against GAPDH and 14-3-3 gamma.

FIG. 4 Results of experiment treating Hep2G cells with 5-aza-2-deoxycytidine in relation to presenilin 1 expression levels normalised against GAPDH and 14-3-3 gamma.

FIG. 5 Results of experiment treating Hep2G cells with vitamins B12 & B6 in relation to MAO-B expression levels normalised against GAPDH and 14-3-3 gamma.

FIG. 6 Results of experiment treating Hep2G cells with 5-aza-2-deoxycytidine in relation to presenilin 1 expression levels normalised against GAPDH and 14-3-3 gamma.

### SUMMARY OF THE INVENTION

The invention, underpinned by the finding that the concentration of MAO-B in patient samples is affected by the SAM methylation cycle, describes the utility of SAM and compounds in the One-carbon cycle in disease treatment therapies in which the disease treatment depends upon MAO-B inhibition. Elucidation of the relationship between MAO-B concentrations and the components of the SAM one-carbon methylation cycle also enables methods of measuring the efficacy of a drug of or a drug that affects the SAM one-carbon methylation cycle in in vitro samples taken from patients, especially patients suspected of having or suffering from Alzheimer's Disease, especially late onset Alzheimer's disease.

### DETAILED DESCRIPTION OF THE INVENTION

In a first aspect, the invention describes the use of one or more of the compounds SAM, vitamin B6, vitamin B12, and folate, individually or in any combination, in the treatment or prevention of diseases which are mediated by monoamine oxidase B inhibitors. In a preferred embodiment the compound(s) is/are one or more of SAM, vitamin B6 and B12; SAM is an especially preferred embodiment. A combination of vitamin B6 and B12 is also a preferred embodiment.

A second aspect of the invention is the use as adjunct therapy of one or more of the compounds SAM, vitamin B6, vitamin B12 and folate, individually or in any combination, in the treatment and prevention of diseases which are mediated by monoamine oxidase B inhibitors. A preferred disease to be treated or prevented by the compounds of the invention when used as adjunct therapy is Alzheimer's disease; treatment of late onset Alzheimer's disease is an especially preferred embodiment. Adjunct therapy implies that the one or more compounds can be used in conjunction with other recognised forms of therapy for a specified diseased.

A preferred disease to be treated or prevented by the compounds of the invention whether alone or as adjunct therapy is Alzheimer's disease; treatment of late onset Alzheimer's disease is an especially preferred embodiment.

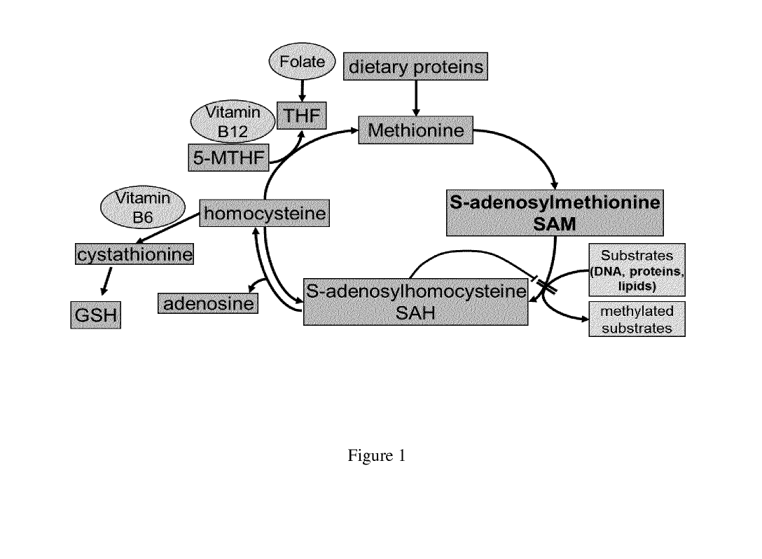

A further aspect of the invention is a method of determining the efficacy of a drug or drugs of the SAM one-carbon methylation cycle for use in therapy comprising measuring in an in vitro sample taken from a patient or from cultured or harvested cells the concentration of MAO-B following administration of a drug or drugs of the SAM one-carbon methylation cycle to the patient or to the cultured or harvested cells and comparing said concentration to a control concentration of MAO-B. The control MAO-B concentration can be a statistical measure of central tendency such as the median or mean of a suitable patient population. Preferably, the control concentration of MAO-B is the concentration of MAO-B in an in vitro sample taken from the patient or from cultured or harvested cells before administration of a drug or drugs. In an embodiment of the invention the MAO-B concentrations measured are normalised using a suitable compound such as GAPDH or 14-3-3 gamma. This aids in minimising the effect of differences in the inter-sample and inter-cellular MAO-B concentrations and can increase result accuracy. In a preferred embodiment the method is used to test the efficacy of drugs of the SAM one-carbon methylation cycle that are used or are to be used in Alzheimer's disease therapy, especially late onset Alzheimer's disease. A concentration of MAO-B in an in vitro sample taken from the patient or from cultured or harvested cells following drug or drugs administration that is lower than that of the control is indicative of an efficacious drug or drugs. What is meant by drug(s) of the SAM one-carbon methylation cycle is a chemical compound that is mentioned in FIG. 1. Representative drugs of the SAM one-carbon methylation cycle include either as monotherapy or in combination: SAM, folate, vitamin B12 and vitamin B6. In a preferred embodiment the drug or drugs of the SAM one-carbon methylation cycle is/are SAM, vitamin B6 and vitamin B12. Most preferably the monotherapeutic drug is SAM, vitamin B6 or vitamin B12 and the combination of drugs is vitamin B6+vitamin B12. If the concentration of MAO-B measured in the in vitro sample taken from the patient or in the cultured or harvested cells is reduced, this denotes that the drug or drugs of the SAM one-carbon methylation cycle is working i.e. the drug(s) is efficacious and can be used for the treatment of AD, preferably LOAD. The skilled person would acknowledge the possible use of numerous cell types for testing drug efficacy; preferred cultured or harvested cells of the method of the invention are HepG2 cells or differentiated neuroblastoma SH-SY5Y cells. The compounds of the SAM one-carbon methylation cycle for all aspects of the invention can be in the form of pharmaceutically acceptable salts.

Alternatively, for pathologies in which an increase in MAO-B concentration is sought, it might be clinically desirable to associate the SAM one-carbon methylation cycle with an increase in MAO-B concentration in a sample taken from a patient. An increase in MAO-B would be expected if the SAM one-carbon cycle were to be interrupted by a drug or drugs causing a decrease in DNA-methylation. A further embodiment of the invention is a method of determining the efficacy of a drug or drugs in a patient to inhibit the SAM one-carbon methylation cycle comprising measuring in an in vitro sample taken from the patient or from cultured or harvested cells the concentration of MAO-B following administration of a drug or drugs to the patient or to the cultured or harvested cells and comparing said concentration to a control concentration of MAO-B. An increase in the concentration of MAO-B in the in vitro patient sample or the cultured or harvested cells compared to control indicates that the SAM one-carbon cycle has been inhibited and that the drug or drugs is/are efficacious and achieve the desired effect.

An additional aspect of the invention is a method of determining the effect of a drug or drugs upon the SAM one-carbon methylation cycle comprising
i. taking an in vitro patient sample or cultured or harvested cells
ii. adding the drug or drugs to be tested
iii. measuring the MAO-B concentration in the in vitro patient sample or cultured or harvested cells
iv. comparing the concentration of MAO-B measured in iii. to a control concentration of MAO-B
   in which a lower MAO-B concentration in the in vitro patient samples or cultured or harvested cells after addition of the drug or drugs compared to the control concentration indicates promotion of the SAM one-carbon methylation cycle, while a greater MAO-B concentration indicates inhibition of the SAM one-carbon methylation cycle. The drug(s) whose effect upon the SAM one-carbon methylation cycle is to be gauged can be naturally occurring molecules or can be man-made synthetic molecules found in or not found in nature. The lower or greater MAO-B concentration of treated samples/cells following addition of drug(s) compared to control is an indirect measure of the degree of DNA-methylation, a greater concentration indicating promotion of DNA methylation. The methods described herein make use of the link that has been elucidated between the SAM one-carbon methylation cycle and the MAO-B concentration and support the evaluation of the efficacy and usefulness of current and future experimental and therapeutic drugs and the use of certain compounds of the SAM one-carbon methylation cycle for treating AD, especially LOAD.

### Methods and Results

### EXAMPLE 1

The neuroblastoma cell line SH-SY5Y was seeded at 110,000 cell/6-well. Neuronal differentiation treatment was started on the next day (=Day 0) by addition of 10 μM retinoic acid every second day until Day 6. On Day 8 differentiation treatment was switched to 4 μM uridine (Ur), 5 μM 5-fluoro-2′-deoxyuridine FdUr), and 1 μM cytosine beta-D-arabinofuranoside (araC). During mitotic inhibitor addition (=Days 8 to 12) cells were left untreated or treated with 625 nM 5-aza-2′-deoxycytidine (Aza), 25 μM S-adenosylmethionine (SAM), 5 μg/ml vitamin B6 (Vit B6), 7.5 μg/ml vitamin B12 (Vit B12), or 5 μg/ml vitamin B6/7.5 μg/ml vitamin B12. On Day 12 cells were harvested. Cells were washed twice with chilled PBS, scraped off in PBS/protease inhibitors, and centrifuged at 15000×g at 4 C for 10 min. The pellet was reconstituted in 150 μl PBS/protease inhibitors and precipitated with 50 μl trichloroacetic acid/80 mMDTT for 1 hour at 4 C. After centrifugation at 15000×g at 4 C for 10 min, precipitated protein was washed four times with 0.6 ml acetone/20 mM DTT and stored at −80 C until analysis. The dried total protein pellet was reconstituted in 7M urea/2M thiourea/4% CHAPS buffer and the protein concentration was determined using the Coomassie Plus (Bradford) Protein Assay (Pierce, Thermo Scientific). Equal protein amounts were loaded onto an 11% SDS polyacrylamide gel and proteins were blotted onto nitrocellulose. Mao-B was detected using a polyclonal Goat anti-Mao-B antibody (sc18401, Santa Cruz) and DyLight 649 AffiniPureF(ab′)2 Fragment Donkey Anti-Goat IgGgamma (Jackson ImmunoResearch Laboratories, Inc.), and a Typhoon scanner (GE Healthcare Life Sciences). Results were analysed with the software ImageJ. The signals of the Mao-B bands of SAM treated and untreated differentiated SH-SY5Y cells were compared and the latter was set 100%.

### EXAMPLE 2

### Treatment with 5-aza-2-deoxycytidine

HepG2 cells were grown in DMEM low glucose/10% FBS and were treated with 10 μM 5-aza-2-deoxycytidine for 1, 2, or 3 days. Untreated HepG2 cells served as controls. Cells were scraped in precipitation buffer (PBS/25% trichloroacetic acid/20 mM DTT/protease inhibitors) at 4 C and proteins were precipitated for 1 hour at 4 C. Precipitated proteins were pelleted at 15000 rpm for 10 min (4 C) and washed 4 times with 700 μl acetone/20 mM DTT. Before analysis by SDS PAGE and Western blot, the precipitated samples were reconstituted in DIGE buffer/1% pH 4-7 ampholyte. Six parallel experiments were performed. Each sample was run on two different Western blots and the results are displayed as averages. Two normalisation compounds were used: GAPDH (glyceraldehyde 3-phosphate dehydrogenase) and 14-3-3 gamma (protein kinase C inhibitor protein 1). Each Mao-B band signal was divided by the respective GAPDH or 14-3-3 gamma band signal to achieve normalisation. As band signals on different Western blots were not comparable due to the use of different antibody solutions, all normalised Mao-B signals of treated cells were compared to the normalised Mao-B signals of the untreated cells, which were set at 100%.

### Treatment with Vitamins B6 and B12

HepG2 cells were grown in DMEM low glucose/2% FBS and were treated with vitamin B6 (300 μg/ml) and vitamin B12 (74.4 μg/ml) for 1, 3, 4, 5 and 7 days. Untreated HepG2 cells served as controls. Cells were harvested and analysed by Western blot.

### Results

TABLE 1
Summary of three experiments: SH-SY5Y cells were differentiated with retinoic
acid and mitotic inhibitors. Cells were treated with different agents, harvested,
and Mao-B protein levels were analysed by Western blot. For each experiment
the Mao-B signals of treated SH-SY5Y cells were compared to untreated cells
(% Mao-B of untreated cells).

| | Densitometric | | | | | | |
|---|---|---|---|---|---|---|---|
| | quantification | | | % MAO-B of untreated cells | | | |
| Treatment | Exp. 1 | Exp. 2 | Exp. 3 | Exp. 1 | Exp. 2 | Exp. 3 | Average |
| Control | 4113.8 | 1136.2 | 1093.1 | 100 | 102 | 98 | 100 |
| Aza | — | 1116.9 | 973.5 | — | 100 | 87 | 94 |
| SAM | 2482.6 | 990.4 | 1006.8 | 60 | 89 | 90 | 80 |
| Vit B6 | 4087.1 | 1008.1 | 1004.8 | 99 | 90 | 90 | 93 |
| Vit B12 | 3823.5 | 857.8 | 695.3 | 93 | 77 | 62 | 77 |
| Vit B6 & B12 | 3425.1 | 852.2 | 585.8 | 83 | 76 | 53 | 71 |

As can be seen from Table 1, SAM, vitamin B6, vitamin B12 and a combination of vitamin B6 and B12 all resulted in a substantial decrease in MAO-B levels compared to the negative control. This suggests a link between the methyl donor S-adenosylmethionine (SAM) and the Mao-B protein expression level via the One-carbon cycle, DNA methyltransferases and MAOB promoter ethylation. Increased availability of SAM, a methyl donor for DNA methyltransferases, may result in elevated MAOB promoter methylation and a decrease in transcription and protein expression of the Mao-B protein. Therefore, SAM appears to regulate Mao-B activity and could be used in therapies of late-onset Alzheimer's disease, Parkinson's disease and other diseases mediated by MAO-B inhibitors. Furthermore, as indicated by the results in Table 1, other compounds of the One-carbon cycle (FIG. 1) are also able to indirectly affect MAO-B levels.

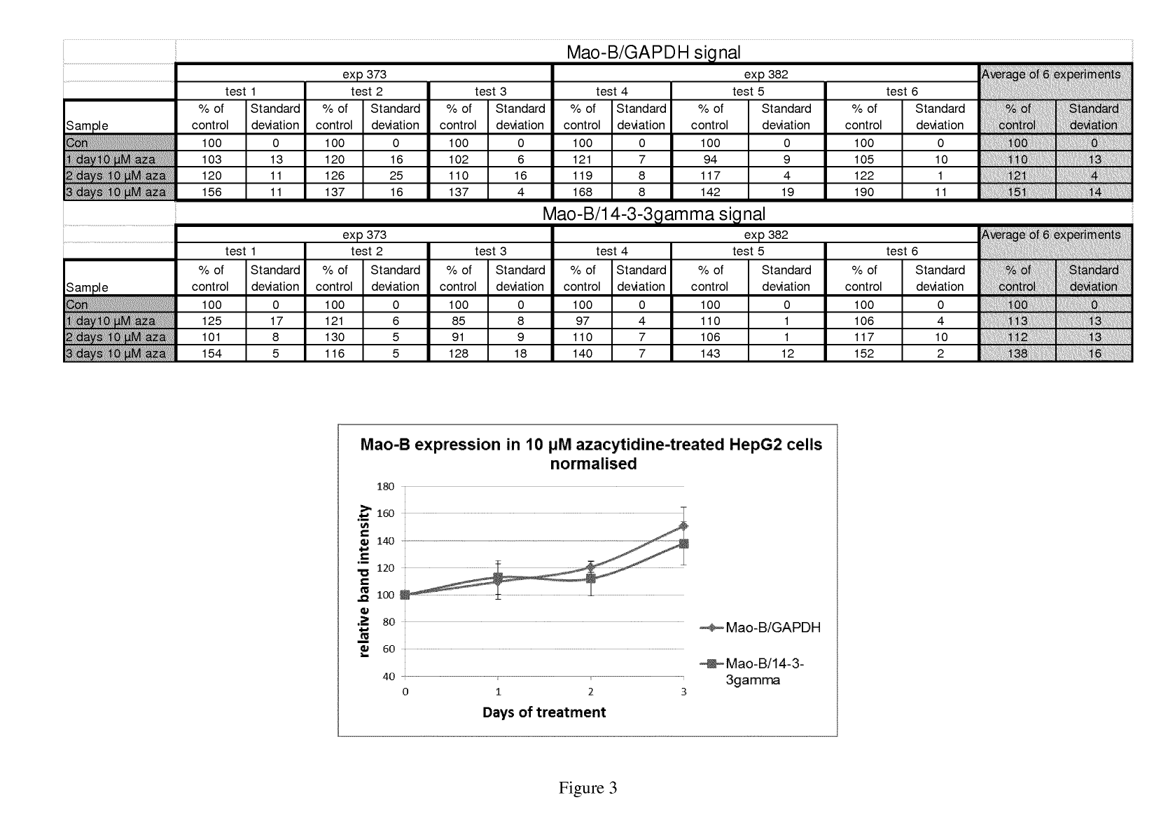

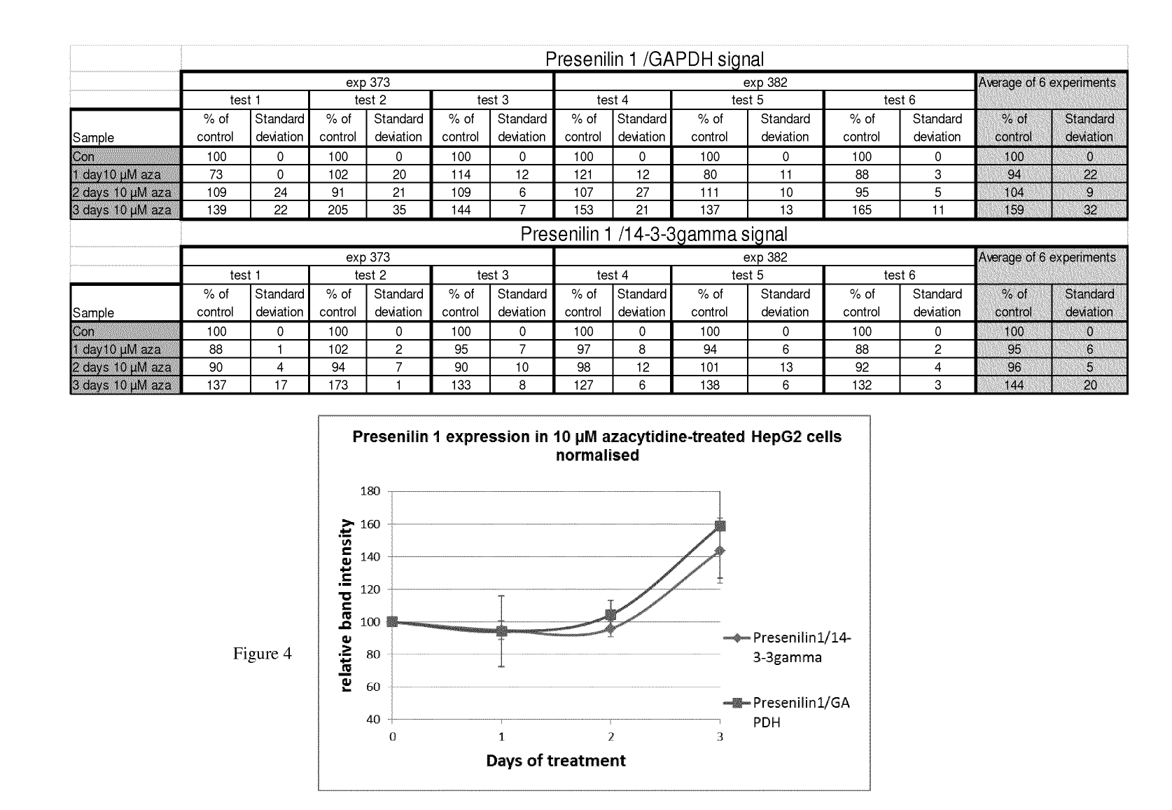

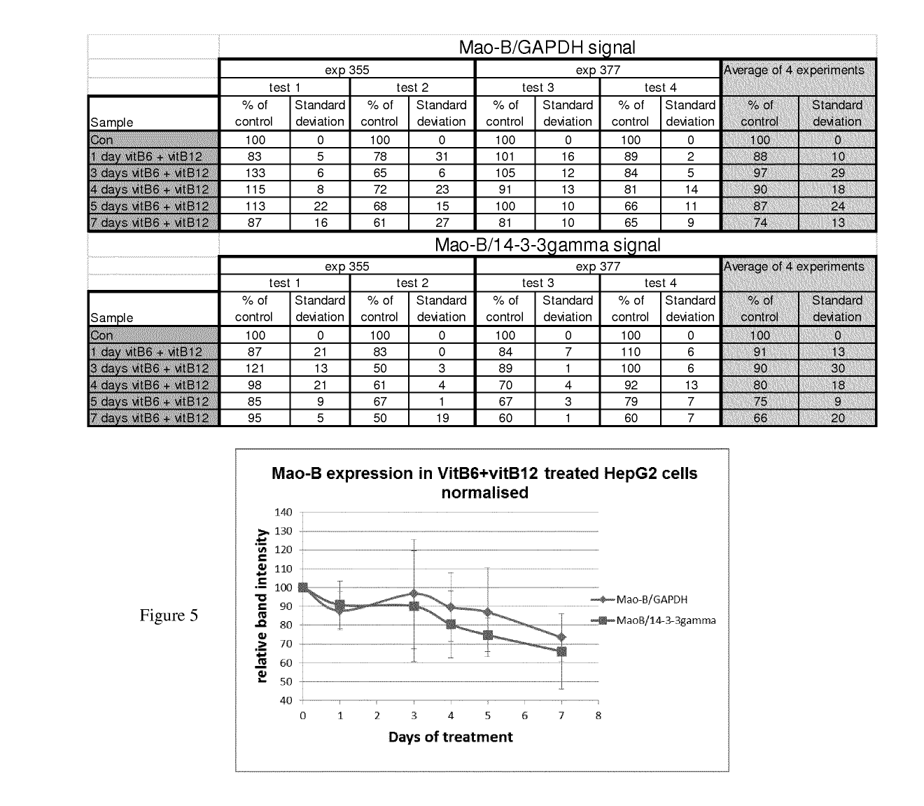

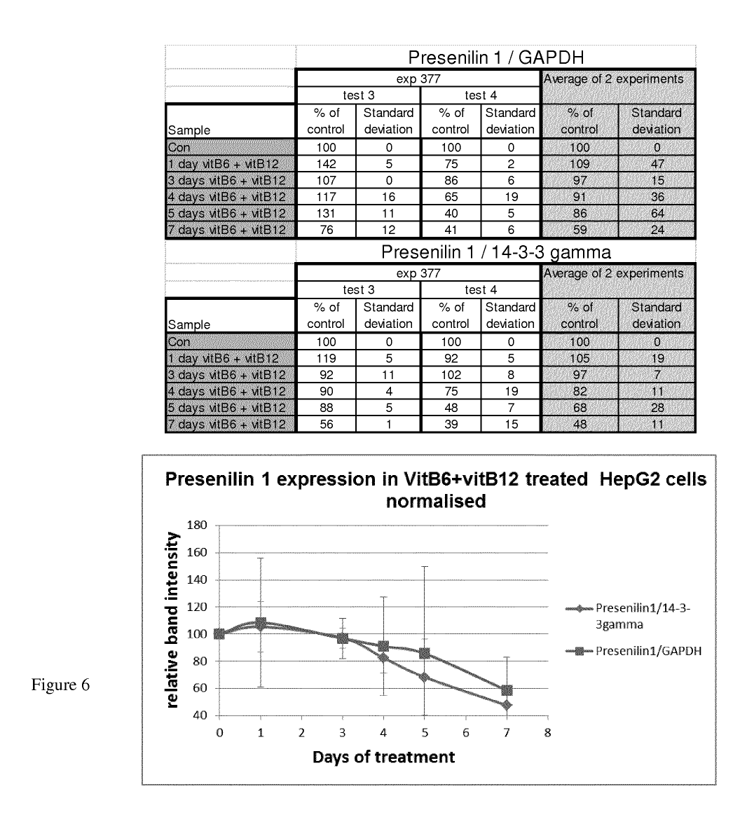

FIGS. 3 to 6 display data that consolidates the physiological connection between the SAM one-carbon cycle and MAO-B levels. FIG. 3 displays the effect of the DNA methyltransferase inhibitor 5-aza-2-deoxycytidine on MAO-B protein expression in HepG2 cells. This data suggests that due to a reduced level of CpG island methylation, increased levels of MAO-B are expressed. FIG. 5 displays the effect of a combination of vitamins B12 & 6 in down-regulating MAO-B protein expression by increasing the supply of SAM and subsequent CpG island methylation increases. FIGS. 4 and 6 display the corresponding effects of 5-aza-2-deoxycytidine and vitamins B12 & B6 on the expression of the AD-related protein preselinin 1; the result of these positive control experiments support the validity of the MAO-B expression experiments and results.

We claim:

1. A method of determining the efficacy of a drug of the SAM one-carbon methylation cycle for therapeutic use comprising measuring in an in vitro sample taken from a patient or from cultured or harvested cells the concentration of monoamine oxidase-B (MAO-B) following administration of a drug of the S-adenosylmethionine (SAM) one-carbon methylation cycle to the patient or to the cultured or harvested cells and comparing said concentration to a control concentration of MAO-B.

2. The method of claim 1, further comprising normalizing the concentration of MAO-B following administration of the drug and the control concentration, using a suitable compound.

3. The method of claim 2, wherein the suitable compound to be used for normalization is glyceraldehyde 3-phosphate dehydrogenase (GAPDH) or 14-3-3-gamma.

4. The method of claim 1, wherein the control concentration of MAO-B is the concentration of MAO-B in an in vitro sample taken from a patient or in cultured or harvested cells before administration of the drug.

5. The method of claim 1, wherein the therapeutic use is the treatment of Alzheimer's disease.

6. The method of claim 5, wherein a concentration of MAO-B measured in an in vitro sample taken from the patient or in cultured or harvested cells following administration of the drug that is lower than that of the control indicates an efficacious drug.

7. The method of claim 5, wherein the drug of the SAM one-carbon methylation cycle is SAM, folate, vitamin B12, vitamin B6, or any combination thereof.

8. The method of claim 7, wherein the drug of the SAM one-carbon methylation cycle is SAM, vitamin B12, vitamin B6, or a combination of vitamin B12 and vitamin B6.

9. A method for treatment or prevention of a disease mediated by monoamine oxidase B inhibition in a patient, comprising administering a compound of the S-adenosylmethionine (SAM) one-carbon methylation cycle to the patient.

10. The method of claim 9, wherein the compound of the SAM one-carbon methylation cycle is S-ademosylmethionine, vitamin B6, vitamin B12, folate, or any combination thereof.

11. The method of claim 10, wherein the compound of the SAM one-carbon methylation cycle is SAM, vitamin B6 and vitamin B12.

12. The method of claim 11, wherein the compound of the SAM one-carbon methylation cycle is a combination of vitamin B6 and vitamin B12.

13. The method of claim 9, wherein the compound of the SAM one-carbon methylation cycle is administered as an adjunct therapeutic drug.

14. The method of claim 9, wherein the disease to be treated or prevented is Alzheimer's disease.

15. The method of claim 9, wherein the disease to be treated is late onset Alzheimer's disease.

16. The method of claim 9, wherein the compound of the SAM one-carbon methylation cycle is in the form of a pharmaceutically acceptable salt.

17. A method of determining the effect of a drug upon the S-adenosylmethionine (SAM) one-carbon methylation cycle, comprising:
i. taking an in vitro patient sample or cultured or harvested cells
ii. adding the drug to be tested;
iii. measuring the monoamine oxidase-B (MAO-B) concentration in the in vitro patient sample or cultured or harvested cells and
iv. comparing the concentration of MAO-B measured in iii. to a control concentration of MAO-B,
wherein a lower MAO-B concentration in the in vitro patient sample or cultured or harvested cells after addition of the drug compared to the control concentration indicates promotion of the SAM one-carbon methylation cycle, and wherein a greater MAO-B concentration in the in vitro patient sample or cultured or harvested cells after addition of the drug compared to the control concentration indicates inhibition of the SAM one-carbon methylation cycle.

18. The method of claim 17, wherein the drug is SAM, vitamin B6, vitamin B12, SAH, methionine, folate, 5-MTHF, THF, cystathionine, homocysteine, GSH, or any combination thereof.

19. The method of claim 18, wherein the drug is SAM, vitamin B6 and vitamin B12.
